(19) United States
(12) Patent Application Publication
(10) Pub. No.: US 2018/0110929 A1
(43) Pub. Date: Apr. 26, 2018

(54) NEEDLELESS INJECTION DEVICE EQUIPPED WITH A COMPRESSION SPRING

(71) Applicant: CROSSJECT, Dijon (FR)

(72) Inventors: Gilles VIVIEN, Malakof (FR); Jean Christophe CLOIX, Longvic (FR)

(73) Assignee: CROSSJECT, Dijon (FR)

(21) Appl. No.: 15/848,043

(22) Filed: Dec. 20, 2017

Related U.S. Application Data
(63) Continuation of application No. PCT/FR2016/051656, filed on Jun. 30, 2016.

(30) Foreign Application Priority Data
Jun. 30, 2015 (FR) 15/56165

(51) Int. Cl. A61M 5/30; A61M 5/20

(52) U.S. Cl. CPC A61M 5/3007; A61M 2005/2013; A61M 5/2033

(57) ABSTRACT

The present disclosure relates to a needleless injector including a cap, an injection system, a body, and a compression spring. The body is mounted to slide upwards in relation to the cap along an injection axis between a rest position and an injection position. The compression spring is axially intercalated along the injection axis between the body and the cap to compress the skin tissue of the user when the nozzle is applied to the skin. The compression spring is a frustroconical helical spring that extends along the injection axis and includes a plurality of turns designed to nest axially one inside the other.

### CROSS-REFERENCE TO RELATED APPLICATIONS

This application is a continuation of International Application No. PCT/FR2016/051656 filed on Jun. 20, 2016, which claims priority to and the benefit of FR 15/56165 filed on Jun. 30, 2015. The disclosures of the above applications are incorporated herein by reference.

### FIELD

The present disclosure relates to a needleless injection device which is equipped with a compression spring.

### BACKGROUND

The statements in this section merely provide background information related to the present disclosure and may not constitute prior art.

The technical field of the present disclosure is one of disposable pre-filled needleless injection devices, operating with an energy source such as a gas generator, and used for the intradermal, subcutaneous and intramuscular injections, of liquid active ingredient for therapeutic use in human or veterinary medicine.

The active ingredient is formed by a more or less viscous liquid, a mixture of liquid, or a gel. The active ingredient may also be a solid dissolved in a solvent suitable for the injection or may be formed of a pulverulent solid suspended at a certain concentration in a suitable liquid. The grain size distribution of the active ingredient must then be compatible with the diameter of the ducts in order to avoid obstructing them.

An injection device includes, in a known manner, for example as in the patent application FR-A-2815544 (equivalent to WO 02/34317), a body comprising successively a gas generator, an expansion chamber, a reservoir containing the liquid active ingredient, and an injection system.

The reservoir is formed by a glass tube which is inserted into a tubular housing defined by the body of the device, and which is obstructed by an upstream plunger and a downstream plunger between which the liquid active ingredient is contained.

The downstream or lower free end of the reservoir cooperates (interfaces) with an injection nozzle which defines at least one injection channel extending axially along an injection axis.

The injection nozzle is axially defined by an upper face bearing axially on the reservoir, and a lower injection face adapted to cooperate with a closure cap.

Furthermore, the injection device includes a hollow cover which envelops the body and which defines a lower opening adapted for the passage of the injection nozzle.

In order to enable the injection of the active ingredient, the body is slidably mounted in the cover, from bottom to top along a sliding axis, between a rest position and an injection position, the driving of the body being carried out when the user presses the injection nozzle on his skin.

The displacement of the body in the cover allows the gas generator to be triggered, generating a pressurized gas which drives in displacement the plungers in order to inject the active ingredient through the user's skin via the injection nozzle.

As shown in the document FR-A-2815544, a helical compression spring is axially interposed, along the sliding axis, between the body and the cover, in order to compress the tissues of the skin of the user during the application of the nozzle on the skin.

The striking device is triggered when the body reaches its injection position. To this end, the user must press on the cover of the injection device, in order to slide the cover relative to the body of the device along a determined triggering travel.

According to this document FR-A-2815544, the triggering travel is given by the sum of the spaces between the turns of the spring. In order to increase this travel, it is desired to increase this space and therefore to increase the total height of the device.

Furthermore, in order to increase the compression force, it is desired to increase the wire diameter of the spring or to add turns, which increases the total height of the device, in particular the height of the cover.

### SUMMARY

The present disclosure aims in particular at overcoming these and other drawbacks and relates for this purpose to a needleless injection device. In one form, the present disclosure includes a cover, an injection system having a plunger, an active ingredient reservoir, an injection nozzle that defines at least one injection channel, a body, a compression spring, a gas generator, and a striking device. The body is enveloped by the cover and is slidably mounted relative to the cover, from bottom to top along an injection axis, between a rest position and an injection position. The compression spring is axially interposed, along the injection axis, between the body and the cover, in order to compress the tissues of the skin of the user during the application of the nozzle on the skin. The striking device is configured to strike the gas generator. The compression spring is a frustoconical-shaped helical spring that extends along the injection axis, and includes a plurality of turns designed to axially nest into each other.

In another form, the compression spring allows reducing the vertical space between the body and the inner face of the cover to a turn thickness of the spring when the body occupies its triggering position.

In yet another form, the compression spring allows varying the calibration of the spring by acting on the section of the wire forming the turns, without substantially reducing the triggering travel of the body.

In one form, due to the frustoconical shape of the spring and to its turns which fit together with little to no axial clearance, the addition of turns may not influence the triggering travel of the body.

In another form, the turns of the spring are contactless with each other during the sliding of the body, so that the spring does not emit friction noise.

According to another form, the compression spring extends axially from a high turn which is bearing on the cover, to a low turn which is bearing on the body, the high turn having a diameter greater than the diameter of the low turn.

In yet another form, the larger diameter of the high turn of the spring allows a better stability of the spring.

According to one form, the cover defines a housing which receives the high turn of the spring, in order to laterally bock in translation the high turn. This allows for a better stability of the spring.

In yet another form, the body forms a boss which protrudes vertically upwards along the injection axis, the low turn of the spring being mounted around the boss, so that the low turn is blocked laterally.

According to another form, the compression spring is calibrated so that the force desired to drive the body from its rest position to its triggering position, and is between five and forty-five Newtons.

In one form, the active ingredient contained in the reservoir is selected from the group consisting of: Methotrexate, Adrenaline, Sumatriptan, Hydrocortisone, Naloxone, Midazolam, Apomorphine, Ethylnatrexone bromide, Phytomenadione, Chlorpromazine hydrochloride, Zuclopenthixol acetate, Danaparoid sodium, Enoxaparin sodium, Estradiol cypionate, Medroxyprogesterone acetate, Medroparin calcium, Methylprednisolone acetate, Heparin calcium, Terbutaline.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 is an axially exploded perspective view, which illustrates a needleless injection device according to the present disclosure;

FIG. 2 is a simplified cross-sectional view, which illustrates the body of the device of FIG. 1 in its rest position; and

FIG. 3 is a cross-sectional view similar to that of FIG. 2, which illustrates the body of the device of FIG. 1 in its triggering position.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

In the description and claims, in order to clarify the description and claims, the longitudinal, vertical and transverse terminology will be adopted by way of non-limiting example with reference to the trihedron L, V, T indicated in the figures.

Furthermore, in the present application, the terms “upper”, “lower”, “horizontal”, “vertical”, and the derivatives thereof refer to the position or orientation of an element or a component, this position or orientation being considered with reference to the orientation of the device in the figures and to the trihedron L, V, T, without reference to the earth's gravity.

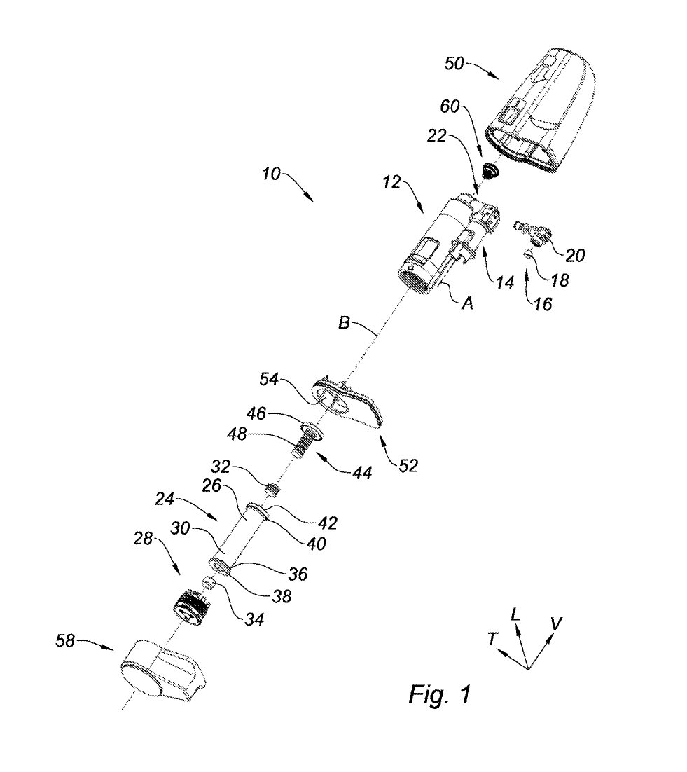

FIG. 1 shows a needleless injection device 10, or needleless syringe which includes a U-shaped body 12 comprising successively a striking device 14, a gas generator 16 comprising a primer 18 and a pyrotechnic charge 20, an expansion chamber 22, a reservoir 24 containing the liquid active ingredient 26 and an injection nozzle 28.

The injection nozzle 28 defines one or more injection channels (e.g. three injection channels (not represented)) and is screwed onto a lower free end of the body 12.

The striking device 14 and the gas generator 16 forms a first linear subassembly of the body 12 that extends axially along a vertical sliding axis A, and the reservoir 24 containing the active ingredient 26 and the injection nozzle 28 form a second linear subassembly of the body 12 that extends axially along a second vertical injection axis B.

These two subassemblies are linked to one another by the expansion chamber 22 that has an axis perpendicular to the axes A, B of the subassemblies.

The reservoir 24 is formed by a glass tube 30 obstructed by an upstream plunger 32 and a downstream plunger 34 between which the active ingredient 26 is contained, the plungers being made of an elastically deformable elastomer-based material.

The reservoir 24 extends axially from a lower collar 36 and has an annular lower face 38 arranged opposite the injection nozzle 28, to an upper collar 40 having an annular upper face 42.

Also, according to FIG. 1, a cylindrical flexible diaphragm 44 includes an annular seat 46 which is axially interposed between the upper face 40 of the reservoir 24 and the outlet orifice of the expansion chamber 22, and a cylindrical body 48 that extends axially inside the reservoir 24, above the upstream plunger 32.

The body 48 of the diaphragm 44 is designed to extend axially, under the effect of the pressure of the gas generated by the gas generator 16, in order to push the upstream plunger 32.

Referring to FIG. 1, the body 12 is enveloped by a hollow cover 50 which delimits or in other words, defines a lower opening closed by a horizontal base plate 52 forming a cover bottom.

The base plate 52 defines a circular passage 54 about the injection axis B which is adapted for the passage and the sliding of the injection nozzle 28 and the downstream end of the body 12, so that the injection nozzle 28 includes a lower section projecting vertically downwards out of the cover 50.

Also, the injection device 10 is equipped with a stopper 58, represented in FIG. 1, and is removably mounted on the body 12 by a bayonet-type locking means.

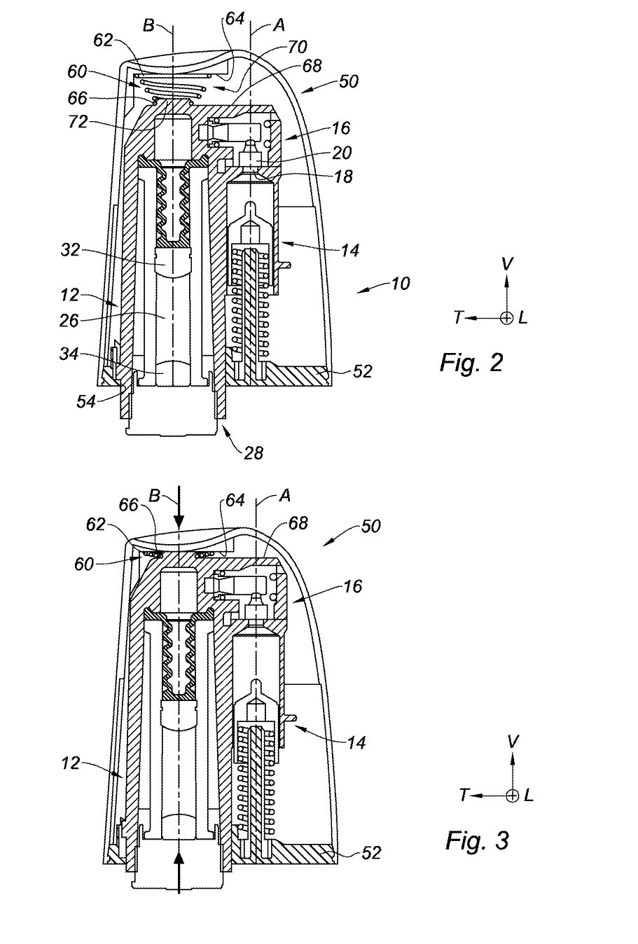

As shown in FIGS. 2 and 3, the stopper 58 is not represented, the body 12 is slidably mounted relative to the cover 50, from bottom to top along the injection axis B, between a rest position illustrated in FIG. 2, and an injection position illustrated in FIG. 3.

The displacement of the body 12 inside the cover 50 allows the gas generator 16 to be triggered, generating a pressurized gas that drives in displacement the plungers 32, 34 in order to inject the active ingredient 26 through the user's skin, via the injection nozzle 28.

Also, the injection device 10 includes a compression spring 60 that is axially interposed, along the injection axis B, between the body 12 and the cover 50, in order to compress the tissues of the skin of the user during the application of the nozzle 28 on the skin, the nozzle 28 being secured in displacement to the body 12.

The compression spring 60 is a frustoconical-shaped helical spring that extends along the injection axis B, and which includes a plurality of turns, here four turns, which are designed to axially nest into each other.

The term “frustoconical-shaped helical spring” means a spring whose turns are adapted to axially nest into each other along the central axis of the spring.

More particularly, the compression spring 60 extends axially from a high turn 62 that is bearing on an inner face 64 of the cover 50, to a low turn 66 that is bearing on an upper face 68 of the body 12. The high turn 62 having a diameter greater than the diameter of the low turn 66.

The cover 50 defines or in other words, defines a housing 70 which receives the high turn 62 of the spring 60, in order to block in translation the high turn 62 in a horizontal plane.

Furthermore, the upper portion of the body 12 forms a boss 72 which protrudes vertically upwards along the injection axis B, from the upper face 68 of the body 12.

The low turn 66 of the spring 60 is mounted around the boss 72, so that the low turn 66 is blocked in translation in a horizontal plane.

According to a preferred variation, the compression spring 60 is calibrated so that the force desired to drive the body 12 from its rest position to its triggering position is comprised between five and forty-five Newtons.

The compression spring 60 according to the disclosure allows reducing the vertical space between the body 12 and the inner face 64 of the cover 50 to a turn thickness of the spring 60, when the body 12 occupies its triggering position, as shown in FIG. 3.

Thus, the spring 60 allows varying the calibration of the spring by acting on the section of the wire forming the turns, without reducing the triggering travel of the body 12.

Furthermore, due to the frustoconical shape of the spring 60 and to its turns which fit together without any axial clearance, the addition of turns does not affect the triggering travel.

Also, in one form, the turns of the spring 60 are contactless with each other during the sliding of the body 12 to inhibit the spring 60 from emitting noise caused by friction.

Furthermore, the larger diameter of the high turn 62 of the spring 60 may improve stability of the spring 60.

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A needleless injection device including:
a cover;
an injection system that comprises a plunger, a reservoir that is configured to hold an active ingredient, and an injection nozzle defining at least one injection channel;
a body that is enveloped by the cover and that is slidably mounted relative to the cover from bottom to top along an injection axis between a rest position and an injection position;
a compression spring that is axially interposed along the injection axis between the body and the cover to compress the tissues of the skin of the user during the application of the nozzle on the skin, wherein the compression spring is a frustoconical-shaped helical spring that extends along the injection axis and includes a plurality of turns designed to axially nest into each other;
a gas generator; and
a striking device that is designed to strike the gas generator.

2. The needleless injection device according to claim 1, wherein the compression spring extends axially from a high turn that bears on the cover to a low turn that bears on the body, the high turn having a diameter greater than the diameter of the low turn.

3. The needleless injection device according to claim 2, wherein the cover defines a housing that receives the high turn of the spring to laterally bock in translation the high turn.

4. The needleless injection device according to claim 2, wherein the body forms a boss that protrudes vertically upwards along the injection axis, the low turn of the spring is mounted around the boss, such that the low turn is blocked laterally.

5. The needleless injection device according to claim 1, wherein the compression spring is calibrated such that the force desired to drive the body from its rest position to its triggering position is between five and forty-five Newtons.

6. The needleless injection device according to claim 1, wherein the active ingredient contained in the reservoir is selected from the group consisting of: Methodtrexate, Adrenaline, Sumatriptan, Hydrocortisone, Naloxone, Midazolam, Apomorphine, Ethylnatrexone bromide, Phytomenadione, Chlorpromazine hydrochloride, Zuclopenthixol acetate, Danaparoid sodium, Enoxaparin sodium, Estradiol cypionate, Medroxyprogesterone acetate, Medroparin calcium, Methylprednisolone acetate, Heparin calcium, Terbutaline.
